(19) United States
(12) Patent Application Publication
(10) Pub. No.: US 2023/0355602 A1
(43) Pub. Date: Nov. 9, 2023

(54) TREATMENT AND/OR PREVENTION OF A VIRAL INFECTION

(71) Applicant: Aslan Pharmaceuticals PTE LTD, Sirgapore (SG)

(72) Inventors: Bertil LINDMARK, Nyhamnslaege (SE); Mark MCHALE, Leicestershire (GB); Ann Gee Lisa OOI, Singapore (SG); Qihui SEET, Sirgapore (SG); Eng Eong OOI, Sirgapore (SG)

(21) Appl. No.: 18/042,920

(22) PCT Filed: Aug. 30, 2021
(86) PCT No.: PCT/SG2021/050523

§ 371 Date: Feb. 24, 2023

(30) Foreign Application Priority Data
Aug. 31, 2020 (SG) 10202008397U

(51) Int. Cl. A61K 31/455; A61P 31/14; A61K 45/06

(52) U.S. Cl. CPC A61K 31/455; A61P 31/14; A61K 45/06

(57) ABSTRACT

A method of treating or preventing a viral infection comprising administering a DHODH inhibitor to a patient in need thereof. Also provided is the use of the DHODH inhibitor for the treatment or prevention of a viral infection, alone or in combination with another therapy, and formulations or compositions comprising the DHODH inhibitor suitable for treating or preventing viral infections.

The present disclosure relates to use of a DHODH inhibitor for the treatment or prevention of a viral infection, alone or in combination with another therapy. The present disclosure further relates to a method of treatment comprising administering the DHODH inhibitor to a patient having or suspected of having a viral infection. Also provided is a method of preventing a viral infection comprising administering the DHODH inhibitor to a patient in need thereof. Further provided are formulations or compositions suitable for treating viral infections.

### BACKGROUND

Viruses are an interesting pathogen because outside a host cell they are unable to replicate. Once they infect host cells, they hijack the cell's machinery to perform viral functions and replicate virions.

Infected host cells, which have been hijacked to perform viral functions have a high metabolic burden.

Dihydroorotate dehydrogenase (DHODH) is the enzyme that catalyzes the fourth step in the pyrimidine biosynthetic pathway namely the conversion of dihydroorotate to orotate concomitantly with an electron transfer to ubiquinone (cofactor Q) via a flavin mononucleotide intermediate (Loffler Mol Cell Biochem, 1997). In contrast to parasites (Plasmodium falciparum) (McRobert et al Mol Biochem Parasitol 2002) and bacteria (E. coli) which exclusively have this de novo pathway as the source of pyrimidines, mammal cells have an additional salvage pathway.

During homeostatic proliferation the salvage pathway, which is independent of DHODH, seems sufficient for the cellular supply with pyrimidine bases. However, in cells with a high turnover the de novo pathway is required to proliferate. In these cells, DHODH inhibition stops the cell cycle progression by suppressing DNA and RNA synthesis and ultimately cell proliferation (Breedveld F. C. Ann Rheum Dis 2000).

There are some suggestions that inhibition of mitochondrial cytochrome bc1, a component of the electron transport chain complex III, leads to activation of p53, followed by apoptosis induction. The mitochondrial respiratory chain is coupled to the de novo pyrimidine biosynthesis pathway via the mitochondrial enzyme dihydroorotate dehydrogenase (DHODH).

Thus, administering a DHODH inhibitor to virally infected cells with a high metabolic burden may inhibit the ability of the virus to replicate, and may promote apoptosis and removal of these cells by the body's natural mechanisms.

Apoptosis is a beneficial form of cell disposal often referred to as programmed cell death and avoids the deleterious effects of necrosis.

The present disclosure provides use of a specific DHODH inhibitor in the treatment or prevention of a viral infection. Advantageously, the mechanism of killing infected cells does not depend on identification/recognition of the virus type or subtype. Thus, the treatment of virus infection according to the present disclosure facilitates and includes variants, even those that are presently unidentified or have not yet emerged.

This is really important because it is clear that vaccination does not necessarily stop infection of cells by the virus and is not necessarily effective against emerging variants.

Whilst not wishing to be bound by theory, there are some hypotheses about viral escape of the immune system. ASLAN003 may be able to stimulate innate immune responses, for example independent of interferon, which could be really important in certain patient populations, including for example vaccinated patients.

### SUMMARY OF THE DISCLOSURE

The present disclosure is summarised in the following paragraphs:
1. A method of treatment comprising administering a therapeutically effective amount of a DHODH inhibitor 2-(3,5-difluoro-3′methoxybiphenyl-4-ylamino)nicotinic acid or a pharmaceutically acceptable salt thereof to a patient with or suspected of having a viral infection.
2. A method of preventing a viral infection comprising administering a therapeutically effective amount of a DHODH inhibitor 2-(3,5-difluoro-3′methoxybiphenyl-4-ylamino)nicotinic acid or a pharmaceutically acceptable salt thereof to a patient in need thereof.
3. A method according to paragraphs 1 or 2, wherein the virus is an RNA virus.
4. A method according to any one of paragraphs 1 to 3, wherein the virus is a single stranded RNA virus.
5. A method according to any one of paragraphs 1 to 4, wherein the virus is a positive-sense, single stranded RNA virus.
6. A method according to any one of paragraphs 1 to 5, wherein the virus is in the category Coronaviridae.
7. A method according to paragraph 6, wherein the virus is selected from Sarbecovirus and Merbecovirus.
8. A method according to paragraph 7, where the virus is Sarbecovirus.
9. A method according to paragraph 8, wherein the virus is selected from SARS-CoV and SARS-CoV-2, in particular SARS-CoV-2 (for example selected from variant Alpha (B.1.1.7), Beta (B.1.351), Gamma (P.1), Delta (B.1.617.2, AY.1, AY.2 & AY.3), Zeta\hat{ } (P.2), Eta (B.1.525 & B.1.1.318), Theta\hat{ } (P.3), Kappa (B.1.617.1, B.1.617.3, AV.1 & C.36.3), Lambda (C37 & B.1.621), Epsilon\hat{ } (B.1.427/B.1.429, B.1.1.7 with S494P & A.27), Iota (B.1.526, B.1.1.7 with Q667H, B.1.620, B.1.214.2, R.1, B.1 with 214insQAS, AT.1, A.30, B.1.630, P.1+N501T and E484Q, B.1.619, B.1.629, C.1.2, B.1.630 & B.1.631/B.1.628) and combinations of two or more of the same).
10. A method according to paragraph 7, wherein the virus is MERS-CoV.
11. A method according to any one of paragraphs 1 to 5, wherein the virus is in the family Flaviviridae.
12. A method according to paragraph 11, wherein the virus is from the genus Flavivirus.
13. A method according to paragraphs 11 or 12, wherein the virus is selected from Dengue virus and Zika virus.
14. A method according to any one of paragraphs 11 to 13, wherein the Dengue virus is serotype 1, 2, 3 or 4.
15. A method according to any one of paragraphs 11 to 13, wherein the virus is Zika virus.
16. A method according to any one of paragraphs 1 to 5, wherein the virus is in the genus alphavirus.
17. A method according to paragraph 16, wherein the virus is selected from Chikungunya virus, Bebaru virus, Getah virus, Mayaro virus, O'nyong'nyong virus, Ross River virus and Semliki Forest virus.
18. A method according to paragraphs 16 or 17, wherein the virus is Chikungunya virus.
19. A method according to any one of paragraphs 1 to 18, wherein the virus is selected from the group comprising a Sarbecovirus and a mosquito borne virus.
20. A method according to paragraph 19, wherein the Sarbecovirus is SARS-CoV or SARS-CoV-2, in particular SARS-CoV-2 (for example selected from variant Alpha (B.1.1.7), Beta (B.1.351), Gamma (P.1), Delta (B.1.617.2, AY.1, AY.2 & AY.3), Zeta\hat{ } (P.2), Eta (B.1.525 & B.1.1.318), Theta\hat{ } (P.3), Kappa (B.1.617.1, B.1.617.3, AV.1 & C.36.3), Lambda (C37 & B.1.621), Epsilon\hat{ } (B.1.427/B.1.429, B.1.1.7 with S494P & A.27), Iota (B.1.526, B.1.1.7 with Q667H, B.1.620, B.1.214.2, R.1, B.1 with 214insQAS, AT.1, A.30, B.1.630, P.1+N501T and E484Q, B.1.619, B.1.629, C.1.2, B.1.630 & B.1.631/13.1.628) and combinations of two or more of the same).
21. A method according to paragraphs 19 or 20, wherein the mosquito borne virus is selected from the group comprising Dengue (such as Dengue serotype 1, 2, 3 or 4), Zika and Chikungunya.
22. A method according to any one of paragraphs 1 to 21, wherein the virus is selected from the group comprising SARS-CoV-2, Dengue (such as Dengue serotype 1, 2, 3 or 4), Zika and Chikungunya.
23. A method according to any one of paragraphs 1 to 22, wherein the patient is human.
24. A method according to paragraph 23, wherein the patient has an age of at least 40 years, for example at 45, at least 50, at least 55, at least 60, at least 65, at least 70, at least 75.
25. A method according to any one of paragraphs 1 to 24, wherein the patient has co-morbidities.
26. A method according to paragraph 25, wherein the co-morbidity is selected from obesity, allergy, asthma, COPD, diabetes, kidney failure, heart disease (including heart failure), cancer, dementia, liver disease, and combinations thereof.
27. A method according to any one of paragraphs 1 to 26, wherein the patient has an ethnicity selected from: Asian, Black and combinations thereof.
28. A method according to any one of paragraphs 1 to 27, wherein the DHODH inhibitor is employed in combination with a further therapy.
29. A method according to paragraph 28, wherein the further therapy is anti-viral and/or anti-inflammatory.
30. A method according to paragraph 29, wherein the further therapy is anti-inflammatory.
31. A method according to paragraph 30, wherein the anti-inflammatory is a steroid.
32. A method according to paragraph 31, wherein the steroid is dexamethasone, for example wherein each dose is in the range 0.5 to 10 mg per day.
33. A method according to any one of paragraphs 28 to 32, wherein the further therapy is anti-viral.
34. A method according to paragraph 33, wherein the anti-viral therapy is remdesivir, for example a first dose of 200 mg followed by daily doses of 100 mg, in particular where the treatment is for 5 to 10 days.
35. A method according to any one of paragraphs 28 to 34, wherein the further therapy is selected from chloroquine, hydroxychloroquinine and pharmaceutically acceptable salts of any one of the same.
36. A method according to any one of paragraphs 1 to 35, wherein the DHODH inhibitor is administered at a dose in the range 1 mg to 400 mg per day, for example 100 mg to 400 mg per day, such as 100, 200, 300 or 400mg per day.
37. A DHODH inhibitor 2-(3,5-difluoro-3′methoxybiphenyl-4-ylamino)nicotinic acid or a pharmaceutically acceptable salt thereof for use in the treatment of a patient with or suspected of having a viral infection.
38. A DHODH inhibitor 2-(3,5-difluoro-3′methoxybiphenyl-4-ylamino)nicotinic acid or a pharmaceutically acceptable salt thereof for use in the prevention of a viral infection in a patient in need thereof
39. A DHODH inhibitor for use according to paragraphs 37 or 38, wherein the virus is an RNA virus.
40. A DHODH inhibitor for use according to any one of paragraphs 37 to 39, wherein the virus is a single stranded RNA virus.
41. A DHODH inhibitor for use according to any one of paragraphs 37 to 40, wherein the virus is a positive-sense, single stranded RNA virus.
42. A DHODH inhibitor for use according to any one of paragraphs 37 to 41, wherein the virus is in the family Coronaviridae.
43. A DHODH inhibitor for use according to paragraph 42, wherein the virus is selected from Sarbecovirus and Merbecovirus.
44. A DHODH inhibitor for use according to paragraph 43, where the virus is Sarbecovirus.
45. A DHODH inhibitor for use according to paragraph 44, wherein the virus is selected from SARS-CoV and SARS-CoV-2 (for example selected from variant Alpha (B.1.1.7), Beta (B.1.351), Gamma (P.1), Delta (B.1.617.2, AY.1, AY.2 & AY.3), Zeta\hat{ } (P.2), Eta (B.1.525 & B.1.1.318), Theta\hat{ } (P.3), Kappa (B.1.617.1, B.1.617.3, AV.1 & C.36.3), Lambda (C37 & B.1.621), Epsilon\hat{ } (B.1.427/B.1.429, B.1.1.7 with S494P & A.27), Iota (B.1.526, B.1.1.7 with Q667H, B.1.620, B.1.214.2, R.1, B.1 with 214insQAS, AT.1, A.30, B.1.630, P.1+N501T and E484Q, B.1.619, B.1.629, C.1.2, B.1.630 & B.1.631/B.1.628) and combinations of two or more of the same).
46. A DHODH inhibitor for use according to paragraph 42, wherein the virus is MERS-CoV.
47. A DHODH inhibitor for use according to any one of paragraphs 37 to 46, wherein the virus is in the family Flaviviridae.
48. A DHODH inhibitor for use according to paragraph 47, wherein the virus is from the genus Flavivirus.
49. A DHODH inhibitor for use according to paragraphs 47 or 48, wherein the virus is selected from Dengue virus and Zika virus.
50. A DHODH inhibitor for use according to any one of paragraphs 47 to 49, wherein the Dengue virus is serotype 1, 2, 3 or 4.
51. A DHODH inhibitor for use according to any one of paragraphs 47 to 49, wherein the virus is Zika virus.
52. A DHODH inhibitor for use according to any one of paragraphs 37 to 41, wherein the virus is in the genus alphavirus.
53. A DHODH inhibitor for use according to paragraph 52, wherein the virus is selected from Chikungunya virus, Bebaru virus, Getah virus, Mayaro virus, O'nyong'nyong virus, Ross River virus and Semliki Forest virus.
54. A DHODH inhibitor for use according to paragraphs 52 or 53, wherein the virus is Chikungunya virus.
55. A DHODH inhibitor for use according to any one of paragraphs 37 to 54, wherein the virus is selected from the group comprising a Sarbecovirus and a mosquito borne virus.
56. A DHODH inhibitor for use according to paragraph 55, wherein the Sarbecovirus is SARS-CoV or SARS-CoV-2, in particular SARS-CoV-2.
57. A DHODH inhibitor for use according to paragraphs 55 or 56, wherein the mosquito borne virus is selected from the group comprising Dengue (such as Dengue serotype 1, 2, 3 or 4), Zika and Chikungunya.
58. A DHODH inhibitor for use according to any one of paragraphs 37 to 57, wherein the virus is selected from the group comprising SARS-CoV-2, Dengue (such as Dengue serotype 1, 2, 3 or 4), Zika and Chikungunya.
59. A DHODH inhibitor for use according to any one of paragraphs 37 to 58, wherein the patient is human.
60. A DHODH inhibitor for use according to paragraph 59, wherein the patient has an age of at least 40 years, for example at 45, at least 50, at least 55, at least 60, at least 65, at least 70, at, least 75.
61. A DHODH inhibitor for use according to any one of paragraphs 37 to 60, wherein the patient has co-morbidities.
62. A DHODH inhibitor for use according to paragraph 61, wherein the co-morbidity is selected from obesity, allergy, asthma, COPD, diabetes, kidney failure, heart disease (including heart failure), cancer, dementia, liver disease, and combinations thereof.
63. A DHODH inhibitor for use according to any one of paragraphs 37 to 62, wherein the patient has an ethnicity selected from: Asian, Black and combinations thereof.
64. A DHODH inhibitor for use according to any one of paragraphs 37 to 63, wherein the DHODH inhibitor is employed in combination with a further therapy.
65. A DHODH inhibitor for use according to paragraph 64, wherein the further therapy is anti-viral and/or anti-inflammatory.
66. A DHODH inhibitor for use according to paragraph 65, wherein the further therapy is anti-inflammatory.
67. A DHODH inhibitor for use according to paragraph 66, wherein the anti-inflammatory is a steroid.
68. A DHODH inhibitor for use according to paragraph 67, wherein the steroid is dexamethasone for example wherein each dose is in the range 0.5 to 10 mg per day.
69. A DHODH inhibitor for use according to any one of paragraphs 64 to 68, wherein the further therapy is anti-viral.
70. A DHODH inhibitor for use according to claim 69, wherein the anti-viral therapy is remdesivir, for example a first dose of 200 mg followed by daily doses of 100 mg, in particular where the treatment is for 5 to 10 days.
71. A DHODH inhibitor for use according to any one of paragraphs 64 to 70, wherein the further therapy is selected from cholorquinine, hydroxxchloroquinine and pharmaceutically acceptable salts of any one of the same
72. A DHODH inhibitor for use according to any one of paragraphs 38 to 71, wherein the DHODH inhibitor is administered at a dose in the range 1 mg to 400 mg per day, for example 100 mg to 400 mg per day, such as 100, 200, 300 or 400 mg per day.
73. Use of a DHODH inhibitor 2-(3,5-difluoro-3′methoxybiphenyl-4-ylamino)nicotinic acid or a pharmaceutically acceptable salt thereof in the manufacture of a medicament for the treatment of a patient with or suspected of having a viral infection.
74. Use of a DHODH inhibitor 2-(3,5-difluoro-3′methoxybiphenyl-4-ylamino)nicotinic acid or a pharmaceutically acceptable salt thereof in the manufacture of a medicament for the prevention of a viral infection in a patient in need thereof.
75. Use according to paragraph 74, wherein the virus is an RNA virus.
76. Use according to paragraph 73 or 74 wherein the virus is a single stranded RNA virus.
77. Use according to any one of paragraphs 74 to 76, wherein the virus is a positive-sense, single stranded RNA virus.
78. Use according to any one of paragraphs 74 to 77, wherein the virus is in the family Coronaviridae.
79. Use according to paragraph 78, wherein the virus is selected from Sarbecovirus and Merbecovirus.
80. Use according to paragraph 79, where the virus is Sarbecovirus.
81. Use according to paragraph 80, wherein the virus is selected from SARS-CoV and SARS-CoV-2, in particular SARS-CoV-2, (for example selected from variant Alpha (B.1.1.7), Beta (B.1.351), Gamma (P.1), Delta (B.1.617.2, AY.1, AY.2 & AY.3), Zeta\hat{ } (P.2), Eta (B.1.525 & B.1.1.318), Theta\hat{ } (P.3), Kappa (B.1.617.1, B.1.617.3, AV.1 & C.36.3), Lambda (C37 & B.1.621), Epsilon\hat{ } (B.1.427/B.1.429, B.1.1.7 with S494P & A.27), Iota (B.1.526, B.1.1.7 with Q667H, B.1.620, B.1.214.2, R.1, B.1 with 214insQAS, AT.1, A.30, B.1.630, P.1+N501T and E484Q, B.1.619, B.1.629, C.1.2, B.1.630 & B.1.631/B.1.628) and combinations of two or more of the same).
82. Use according to paragraph 79, wherein the virus is MERS-CoV.
83. Use according to any one of paragraphs 74 to 77, wherein the virus is in the family Flaviviridae.
84. Use according to paragraph 83, wherein the virus is from the genus Flavivirus.
85. Use according to paragraphs 83 or 84, wherein the virus is selected from Dengue virus and Zika virus.
86. Use according to any one of paragraphs 83 to 85, wherein the Dengue virus is serotype 1, 2, 3 or 4.
87. Use according to any one of paragraphs 83 to 86, wherein the virus is Zika virus.
88. Use according to any one of paragraphs 74 to 77, wherein the virus is in the genus alphavirus.
89. Use according to paragraph 88, wherein the virus is selected from Chikungunya virus, Bebaru virus, Getah virus, Mayaro virus, O'nyong'nyong virus, Ross River virus and Semliki Forest virus.
90. Use according to paragraphs 88 or 89, wherein the virus is Chikungunya virus.
91. Use according to any one of paragraphs 74 to 90, wherein the virus is selected from the group comprising a Sarbecovirus and a mosquito borne virus.
92. Use according to paragraph 91, wherein the Sarbecovirus is SARS-CoV or SARS-CoV-2, in particular SARS-CoV-2 (for example selected from variant Alpha (B.1.1.7), Beta (B.1.351), Gamma (P.1), Delta (B.1.617.2, AY.1, AY.2 & AY.3), Zeta\hat{ } (P.2), Eta (B.1.525 & B.1.1.318), Theta\hat{ } (P.3), Kappa (B.1.617.1, B.1.617.3, AV.1 & C.36.3), Lambda (C37 & B.1.621), Epsilon\hat{ } (B.1.427/B.1.429, B.1.1.7 with S494P & A.27), Iota (B.1.526, B.1.1.7 with Q667H, B.1.620, B.1.214.2, R.1, B.1 with 214insQAS, AT.1, A.30, B.1.630, P.1+N501T and E484Q, B.1.619, B.1.629, C.1.2, B.1.630 & B.1.631/B.1.628) and combinations of two or more of the same).
93. Use according to paragraphs 91 or 92, wherein the mosquito borne virus is selected from the group comprising Dengue (such as Dengue serotype 1, 2, 3 or 4), Zika and Chikungunya.
94. Use according to any one of paragraphs 74 to 93, wherein the virus is selected from the group comprising SARS-CoV-2, Dengue (such as Dengue serotype 1, 2, 3 or 4), Zika and Chikungunya.
95. Use according to any one of paragraphs 74 to 94, wherein the patient is human.
96. Use according to paragraph 95, wherein the patient has an age of at least 40 years, for example at 45, at least 50, at least 55, at least 60, at least 65, at least 70, at least 75.
97. Use according to any one of paragraphs 74 to 96, wherein the patient has co-morbidities.
98. Use according to paragraph 97, wherein the co-morbidity is selected from obesity, allergy, asthma, COPD, diabetes, kidney failure, heart disease (including heart failure), cancer, dementia, liver disease, and combinations thereof.
99. Use according to any one of paragraphs 74 to 98, wherein the patient has an ethnicity selected from: Asian, Black and combinations thereof.
100. Use according to any one of paragraphs 74 to 99, wherein the DHODH inhibitor is employed in combination with a further therapy.
101. Use according to paragraph 100, wherein the further therapy is anti-viral and/or anti-inflammatory.
102. Use according to paragraph 101, wherein the further therapy is anti-inflammatory.
103. Use according to paragraph 102, wherein the anti-inflammatory is a steroid.
104. Use according to paragraph 101, wherein the steroid is dexamethasone, for example wherein each dose is in the range 0.5 to 10 mg per day.
105. Use according to any one of paragraphs 100 to 104, wherein the further therapy is anti-viral.
106. Use according to paragraph 105 wherein the anti-viral therapy is remdesivir, for example a first dose of 200 mg followed by daily doses of 100 mg, in particular where the treatment is for 5 to 10 days.
107. Use according to any one of claims 105 to 106, wherein the further therapy is selected from chloroquine, hydroxychloroquinine and pharmaceutically acceptable salts of any one of the same
108. Use according to any one of paragraphs 71 to 104, wherein the DHODH inhibitor is administered at a dose in the range 1 mg to 400 mg per day, for example 100 mg to 400 mg per day, such as 100, 200, 300 or 400 mg per day.

Thus, in one aspect, there is provided a method of treating or preventing a viral infection, comprising administering a therapeutically effective amount of a DHODH inhibitor 2-(3,5-difluoro-3′methoxybiphenyl-4-ylamino)nicotinic acid or a pharmaceutically acceptable salt thereof to a patient in need thereof.

In one embodiment, the method is for treating a patient with or suspected of having a viral infection.

In one embodiment the DHODH inhibitor of the present disclosure is employed in combination with one or more vaccines, for example one or more SARS-CoV-2 vaccines. Thus in one embodiment the treatment is in a patient population that is vaccinated.

In one embodiment the treatment is in a patient population that is unvaccinated.

In one embodiment the treatment is in a patient population that is partially vaccinated, for example a patient has received the first vaccine but has not received the requisite booster.

The present inventors have established that the DHODH inhibitor 2-(3,5-difluoro-3′methoxybiphenyl-4-ylamino)nicotinic acid (also known as ASLAN003) is efficacious against a range of viruses, including SARS-CoV-2, Zika, Dengue and Chikugunya, where it has demonstrated efficacy with sub-micromolar potency. The use of ASLAN003 in treating a viral infection has a potential three-fold effect: (a) to inhibit viral replication, (b) to activate the innate immune response independent of interferon, and (c) to inhibit the inflammatory response associated with virus infection.

The ability of ASLAN003 to inhibit the inflammatory response may be particularly helpful for viral infections such as SARs and COVID-19, which are characterised by an overexuberant inflammatory response. Hence, because of its triple action, using ASLAN003 to treat or prevent a viral infection may be beneficial over other conventional anti-viral agents which are typically only able to inhibit viral replication. Furthermore, because DHODH is a host cell target, it is agnostic to virus mutation offering protection from viral resistance.

Thus, in one embodiment ASLAN003 reduces the presence of pro-inflammatory cytokines, in comparison to where the drug is not administered.

In one embodiment ASLAN003 inhibits proliferation of cells with inappropriate levels of metabolic activity.

Advantageously ASLAN003 leaves healthy cells unaffected.

DHODH inhibitors have different profiles and mechanisms of action. Thus, it is difficult to predict in advance how they will work in a given situation without testing them.

In one embodiment ASLAN003 depletes the nucleotide pool in an infected cell and blocks virus genome synthesis.

In one embodiment cellular stress in an infected cells treated with ASLAN003 stimulates innate immune response, for example in particular patient populations.

In one embodiment the use of ASLAN003 inhibits hyperactive immune cells and thereby blocks inappropriate inflammatory responses. Inappropriate inflammatory responses can lead to the induction of cytokine storm. The latter can be really dangerous in patients and at the present time there are no really effective ways of preventing/treating this in patients. Administering ASLAN003 at an appropriate stage of infection may mitigate the induction of cytokine storm.

In one embodiment use of ASLAN003 reduces the need (duration or amount of) of oxygen support required by a patient.

In one embodiment use of ASLAN003 may provide in a patient improvements in one or more of the following: Time to Respiratory Improvement; Rate of viral elimination (for example at day 14 and 28); Time to viral elimination; Time to discharge from hospital; Reduction in duration of intensive care treatment; and/or Reduction in Viral Load.

In the context of “Time to Respiratory Improvement”; Respiratory improvement as employed herein refers to sustained peripheral oxygen saturation (SpO2)≥94% on room air.

Rate of viral elimination at Day 14 and Day 28 as employed herein refers to the proportion of patients with undetectable SARS-CoV-2 RNA level on Day 14 and Day 28.

Time to viral, elimination [Time Frame: 28 Days] as employed herein is defined as time (in days) from randomization to undetectable SARS-CoV-2 RNA level.

Time to Discharge from hospital as employed herein refers to the period between when the patient is first admitted to hospital and when the patient is discharged from hospital [example time frame: 28 Days].

Reduction in Viral Load as employed herein refers to a reduction in the number of viral particles in a patient's blood over a period of time [for example up to Day 28].

In one embodiment ASLAN003 is administered orally, for example as a tablet or capsule. This is advantageous because it does not require a loading dose or complicated dosing regimen.

Advantageously, the use of ASLAN003 for treating a viral infection has surprising and useful technical benefits over other known DHODH inhibitors. For example, ASLAN003 is well tolerated in patients:

In a phase 2 clinical study in acute myeloid leukaemia (AML) patients, (many of whom are extremely ill) ASLAN003 was safe and well-tolerated with no evidence of significant liver enzyme rises. Full exposure is achieved within 24 hours and a half-life of 18 hours allows for rapid clearance on cessation of treatment, and PK profiles in Asian and Caucasian patients were equivalent.

Being an oral therapy with no requirements for loading dose, ASLAN003 offers a simple dosing regimen of once or twice daily treatment which could also have utility in a post-contact or outpatient setting. Data from the ASLAN003 Phase 2 AML study support flexible dosing schedules from 100 mg to 400 mg, which can be tailored to the patient's individual needs and the intended dose.

In contrast, DHODH inhibitors such as leflunomide and Brequinar, suffer from severe liver toxicities and other side effects. In comparison, a recent study demonstrated that ASLAN003 has a superior safety profile compared to leflunomide, teriflunomide, vidofludimus and brequinar (Jones et al, Toxicology in Vitro 72(2021), 105096).

In addition, ASLAN003 is a substantially more potent DHODH inhibitor compared to other DHODH inhibitors. See Table 1 below:

TABLE 1
ASLAN003 is a substantially more potent DHODH
inhibitor than other DHODH inhibitors

| | ASLAN003² | Brequinar³ | Vidofludimus¹ | Teriflunomide⁴ | Leflunomide³ |
|---|---|---|---|---|---|
| hDHODH IC₅₀ | 0.035 | 0.010 | 0.12 | 1.3 | 98 |
| (μM) | | | | | |

¹Immunic Therapeutics website and press release
²ASLAN Pharmaceuticals website and publications
³Doi: 10.1016/s0006-2952(98)00145-2
⁴FDA website. Aubagio published tertiary pharmacology review (202992Orig1s000)

Hence, ASLAN003 has an optimised activity profile that renders it particularly suitable for use in the treatment of viral infections.

Further advantageously, ASLAN003 is a very stable drug, which has been demonstrated at elevated temperatures. This is beneficial because in hot climates in non-industrialised countries, where viral infections are prolific and access to electricity (and hence refrigeration) is limited, the high stability of ASLAN003 makes it a good candidate for a drug which does not rely on cold chain transport.

Furthermore, the stability of ASLAN003 makes it amenable to long term stockpiling, which is relevant in all countries. In comparison, although other anti-viral drugs such as Tamiflu are stockpiled, the requirement for refrigerated storage and their typically short shelf life mean that such anti-viral agents tend to be expensive and impractical to store. This can lead to wastage because stocks need to be destroyed if they expire before they are used, and inefficient, because stocks have to be continuously replenished.

Lastly, from a manufacturing perspective, ASLAN003 has a relatively simple chemical structure which helps make it cheaper to manufacture on large scale—this is particularly attractive to poorer countries as it means that price should not be a barrier to access.

In summary, the use of ASLAN003 to treat or prevent a viral infection has numerous advantages:
i. ASLAN003 provides a multi-pronged approach to the treatment of a viral infection: it inhibits viral replication, it activates the innate immune response independent of interferon, and it inhibits the inflammatory response;
ii. ASLAN003 inhibits DHODH, which is a host cell target, thus it is less susceptible to anti-viral resistance;
iii. ASLAN003 is highly potent and has a better safety profile than competing DHODH inhibitors;
iv. ASLAN003 is very stable and cheap to manufacture, making it easy to store and stockpile, which is ideal for use in resource poor environments.

### BRIEF SUMMARY OF THE FIGURES

FIG. 1 shows an IC50 curve for ASLAN003

FIGS. 2A & B shows cell viability of A549 cells when treated with ASLAN003. (A) Experiment 1, (B) Experiment 2. Note there is no change in A549 cell viability when treated with ASLAN003.

FIG. 3 shows results of Huh7 cells that have been infected with Dengue virus serotype 1 (DENV1) when treated with ASLAN003

FIG. 4 shows results of Huh7 cells that have been infected with Dengue virus serotype 2 (DENV2) when treated with ASLAN003

FIG. 5 shows results of Huh7 cells that have been infected with Dengue virus serotype 3 (DENV3) when treated with ASLAN003

FIG. 6 shows results of Huh7 cells that have been infected with Dengue virus serotype 4 (DENV4) when treated with ASLAN003

FIG. 7 shows results of Huh7 cells that have been infected with Zika virus (ZIKV) when treated with ASLAN003

FIG. 8 shows results of time-of-addition assay for Huh7 cells that have been infected with Dengue virus serotype 2 when treated with ASLAN003

FIG. 9 shows results of Huh7 cells that have been infected with Chikungunya virus (CHIKV) when treated with ASLAN003

FIG. 10 shows results of Huh7 cells that have been infected with Chikungunya virus (CHIKV) when treated with ASLAN003

FIG. 11 shows anti-viral activities of ASLAN003 observed in Vero E6 cells. For evaluation of compounds with antiviral activity, Vero E6 cells were infected with SARS-CoV-2 and treated with increasing concentrations of ASLAN003. 0.1% DMSO is added as the non-treatment control. The primary and secondary axis correspond to the viral titre (bar) and relative cell viability (line), and the dashed line represents the CC80 cut-off for cell viability. One-way ANOVA and Dunnett's post-test was used to determine statistical differences, with* denoting p<0.05 and *** denoting p<0.001. Error bars represent the standard deviation observed from the means of triplicates performed for both cell viability and dose-dependent inhibition studies.

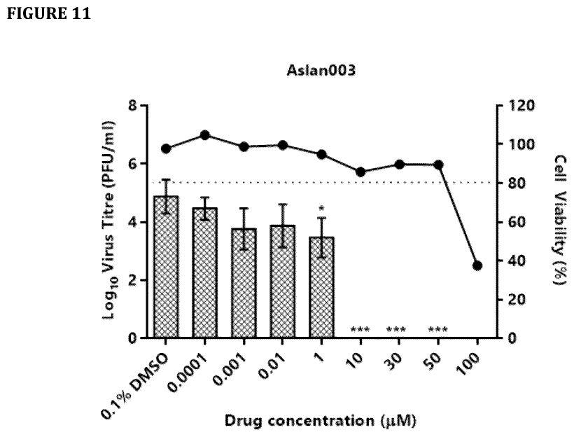

### DETAILED DESCRIPTION

As used herein, virus refers to a virus that is responsible for a viral infection or viral disease. Thus, a virus in the context of the present disclosure generally refers to a pathogenic virus. It is not intended to refer to a viral vector or virus employed as a therapeutic agent.

Viral infection or viral disease as used interchangeably herein refers to an infection or disease caused by the presence of a virus in the body.

In one embodiment, the virus responsible for the viral infection is an RNA virus.

RNA viruses are generally categorised as Riboviria and Orthornavirae. There are single stranded forms and also double stranded forms (group III in the Baltimore classification) of RNA viruses. Single stranded viruses are divided into: positive sense single stranded RNA viruses (group IV Baltimore classification); negative sense single stranded RNA viruses (group V Baltimore classification) and single stranded RNA viruses with a DNA intermediate (group VI Baltimore classification). Hence, in one embodiment, the virus is a single stranded RNA virus, for example a positive sense single stranded RNA virus or a negative sense single stranded RNA virus.

In one embodiment, the virus responsible for the viral infection is Sarbecovirus. Sarbecovirus is the subgenus of viruses that cause severe acute respiratory syndrome. Examples of Sarbecovirus include SARS-CoV, SARS-CoV2, Bat SARS-like coronavirus WIV1 and Bat coronavirus RaTG13. The genus is betacoronavirus and the family is coronaviridae. Accordingly, in one embodiment, the virus is in the Coronaviridae family.

Merbecovirus is the subgenus of viruses that cause Middle East respiratory syndrome-related coronavirus. The genus is betacoronavirus and the family is coronaviridae. Thus, in one embodiment, the virus is Merbecovirus.

The present disclosure also extends to treatment of Embecovirus, (known as group 2a coronavirus) such as Human Coronavirus HKU1. Hence, in one embodiment the virus is a Embecovirus.

The present disclosure also includes Nobecovirus (known as group 2d coronavirus).

In one embodiment, the virus responsible for the viral infection is SARS-CoV. SARS-CoV is also referred to a SARS-CoV1 or SARS.

In one embodiment, the virus is SARS-CoV2. SARS-CoV2 is also referred to a coronavirus or COVID-19.

In one embodiment, the virus is MERS-CoV. MERS-CoV is also referred to a Middle East respiratory syndrome coronavirus.

The viral infection may be a mosquito borne viral disease or due to a mosquito borne virus. Common examples of mosquito-borne diseases include malaria, Chikungunya, Dengue, Zika, West Nile virus, and yellow fever. Yellow, Dengue, and chikungunya are transmitted mostly by Aedes aegypti mosquitoes, Other mosquito-borne diseases like epidemic polyarthritis, Rift Valley fever, Ross River fever, St. Louis encephalitis, West Nile fever, Japanese encephalitis, La Crosse encephalitis are typically carried by several different mosquitoes.

Thus, in one embodiment, the viral infection is a mosquito-borne viral disease. In other words, in one embodiment, the virus is transmitted by mosquitoes.

In one embodiment, the virus is a Flavivirus. Flavivirus is a genus of viruses that include west nile virus, Dengue virus, tick-borne encephalitis virus, yellow fever virus, Zika virus etc. Flaviviridae is the family. Accordingly, in one embodiment the virus is in the Flaviviridae family.

The present disclosure extends to the treatment of Dengue virus. There are 5 serotypes of the virus, all of which are able to cause the full spectrum of disease. In one embodiment, the Dengue virus is any one of serotypes 1 to 5, in particular serotypes 1, 2, 3 or 4. Thus, in one embodiment, the virus is Dengue serotype 1, 2, 3 or 4. In one embodiment, the virus is Dengue serotype 1. In one embodiment, the virus is Dengue serotype 2. In one embodiment, the virus is Dengue serotype 3. In one embodiment, the virus is Dengue serotype 4. In one embodiment, the virus is Dengue serotype 5.

In one embodiment, the virus is an Alphavirus. Alphavirus is a genus of viruses that is the sole genus in the Togaviridae family. There are 31 alphaviruses which include Chikungunya virus, Bebaru virus, Getah virus, Mayaro virus, O'nyong'nyong virus, Ross River virus and Semliki Forest virus.

In one embodiment, the virus is selected from the group comprising a Sarbecovirus and a mosquito borne viral disease.

In one embodiment, the Sarbecovirus is SARS-CoV or SARS-CoV-2, in particular SARS-CoV-2.

In one embodiment, the mosquito borne viral disease or viral infection is selected from the group comprising Dengue, Chikungunya and Zika. In one embodiment, the Dengue is Dengue serotype 1, 2, 3 or 4.

Thus, in one embodiment, the virus is selected from the group comprising SARS-CoV-2, Dengue, Chikungunya and Zika.

In one embodiment, the DHODH inhibitor is 2-(3,5-difluoro-3′-methoxybiphenyl-4-ylamino) nicotinic acid (referred to herein as ASLAN003) or a pharmaceutically acceptable salt thereof, in particular:

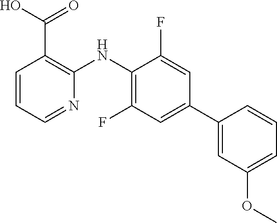 [SMILES: COc1cccc(-c2cc(F)c(Nc3ncccc3C(=O)O)c(F)c2)c1]

In one embodiment the DHODH inhibitor is administered daily, for example once daily.

References to a DHODH inhibitor or salt thereof as employed herein includes providing the compound as a prodrug, such as an ester that is converted to the active ingredient in vivo.

In one embodiment the DHODH inhibitor is administered orally.

Treatment as employed herein refers to where the patient has a disease or disorder, for example viral infection and the medicament according to the present disclosure is administered to stabilise the disease, delay the disease, amelorate the disease, send the disease into remission, maintain the disease in remission or cure the disease. Treating as employed herein includes administration of a medicament according to the present disclosure for treatment or prophylaxis, i.e. for prevention of a viral infection.

In one embodiment treatment/therapy includes prophylactic treatment.

In one embodiment prophylactic treatment is given to a person suspected of having a viral infection.

Prevention of a viral infection as employed herein refers to administering the therapy to a healthy person to prevent them getting a viral infection.

In one embodiment treatment/therapy does not include prophylactic treatment.

Patient as employed herein includes a patient having a viral infection, a patient suspected of having a viral infection, and also a patient who does not have a viral infection yet. In the latter case, the DHODH inhibitor of the present disclosure may be administered in order to prevent the patient from having a viral infection.

A patient having a viral infection as employed herein refers to a patient, identified as having a viral infection by an appropriate test or a patient suspected of having a viral infection.

A DHODH inhibitor is a moiety (such as a compound) that inhibits, for example reduces or blocks the activity of a DHODH enzyme (see background for definition thereof).

Therapeutically effective amount as employed herein is an amount in the range which generates a desirable physiological effect, whilst minimising side effects.

The DHODH inhibitor of the disclosure or formulation comprising the same may be administered at a dose in the range of 1 mg to 400 mg per day, such as 10 mg to 400 mg per day, 50 mg to 400 mg per day, 100 mg to 400 mg per day, 150 mg to 400 mg per day, 200 mg to 400 mg per day, 250 mg to 400 mg per day, 300 mg to 400 mg per day, or 350 mg to 400 mg per day. Thus, in one embodiment therapeutically effective amount of DHODH inhibitor is in the range of 1 mg to 400 mg per day.

In one embodiment, a dose in the range of 100 mg to 400 mg per day is administered.

Thus, in one embodiment the daily dose may be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.

Co-morbidity as employed herein refers to where the patient is suffering from a second or underlying health condition.

In one embodiment the DHODH inhibitor is administered as a combination therapy with a second or further therapy.

Combination therapy (comprising further therapy) as employed herein wherein two or more treatment regimens are employed, in particularly employed concomitantly. The treatments may be separate formulations or co-formulated. They may be administered at the same time or different times. However, the pharmacological effect of the treatments will co-exist in the patient. Further therapy as employed herein refers to a therapy in addition to the DHODH inhibitor.

In one embodiment, the further therapy is an anti-viral therapy.

Anti-viral therapy as used herein, is any therapy employed to reduce or ameliorate viral infection or symptoms associated therewith, in particular viral infection. Examples of anti-viral therapies include but are not limited to Abacavir, Aciclovir, Acyclovir, Adefovir, Amantadine, Amprenavir, Ampligen, Arbidol, Atazanavir, Atripla, Boceprevirertet, Cidofovir, Combivir, Darunavir, Delavirdine, Didanosine, Docosanol, Edoxudine, Efavirenz, Emtricitabine, Enfuvirtide, Entecavir, Entry inhibitors, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir, Peginterferon alfa-2a, Penciclovir, Peramivir, Pleconaril, Podophyllotoxin, Protease inhibitor, Raltegravir, Remdesivir, Reverse transcriptase inhibitor, Ribavirin, Rimantadine, Ritonavir, Pyramidine, Saquinavir, Stavudine, Synergistic enhancer (antiretroviral), Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir and Zidovudine.

In one embodiment, the anti-viral therapy is Remdesivir.

In one embodiment, the further therapy is an anti-inflammatory agent. Anti-inflammatory as employed herein refers to a moiety that reduced inflammation, for example a non-steroidal anti-inflammatory, steroids and the like.

Examples of anti-inflammatory agents include but are not limited to a non-steroidal anti-inflammatory agent (NSAID), a disease modifying anti-rheumatic drug (DMARD), a statin (including HMG-CoA reductase inhibitors such as simvastatin), a biological agent (biologicals), a steroid, an immunosuppressive agent, a salicylate and/or a microbicidal agent.

Non-steroidal anti-inflammatory agents include anti-metabolite agents (such as methotrexate) and anti-inflammatory gold agents (including gold sodium thiomalate, aurothiomalate or gold salts, such as auranofin). Biologicals include anti-TNF agents (including adalimumab, etanercept, infliximab, anti-IL-1 reagents, anti-IL-6 reagents, anti-CD20 agents, anti-B cell reagents (such as rituximab), anti-T cell reagents (anti-CD4 antibodies), anti-IL-15 reagents, anti-CLTA4 reagents, anti-RAGE reagents), antibodies, soluble receptors, receptor binding proteins, cytokine binding proteins, mutant proteins with altered or attenuated functions, RNAi, polynucleotide aptamers, antisense oligonucleotides or omega 3 fatty acids. Steroids (also known as corticosteroids) include cortisone, prednisolone or dexamethasone may also be employed in a combination therapy according to the present disclosure.

Further examples of NSAIDS include salicylates, such as aspirin (acetylsalicyclic acid), diflunisal, salicylic acid and its salts, salsalate (disalcid); proprionic acid derivaties, such as ibuprofen (isobutylphenylpropionic acid), dexibuprofen, naproxen, fenoprofen, ketoprofen, dexketoprofen, flurbiprofen, oxaprozin, loxoprofen, pelubiprofen, zaltoprofen; acetic acid derivatives, such as indomethacin, tolmetin, sulindac, etodolac, ketorolac, diclofenac, aceclofenac, bromfenac, nabumetone; enolic acid (oxicam) derivaties such as piroxicam, meloxicam, tenoxicam, droxicam, lornoxicam, and phenylbutazone (Bute); anthranilic acid derivatives (fenamates), such as mefenamic acid, flufenamic acid, meclofenamic acid and tolfenamic acid; selective COX-2 inhibitors (coxitis), such as celecoxib, parecoxib and etoricoxib; sulfonailides such as nimesulide; clonixin and licofelone.

In one embodiment, the anti-inflammatory agent is an antibody selected from the group comprising an anti-CD3 (such as eplizumab), an anti-IL-2 receptor (such as daclizumab), an anti-CD20 antibody (such as rituximab, crelizumab, ofatumumab), an anti-CD22 (such as epratuzumab), an anti-TNF-α (e.g. infliximab, adulimumab, golimumab), an anti-Blys (such as belimumab), an anti-IL-6 (e.g. MEDI5117), an anti-IL-6 receptor (such as toculizumab), an anti-IL-12/23 (e.g. ustekinumab), an anti-IL-17 (e.g. AIN457/LY24398), an anti-IL-1β (e.g. canakinumab), an anti-IL-1R (such as AMG108), an anti-a4 integrin (e.g, natalizumab) and an anti-LFA-1 (such as efalizumab).

Immunosuppressive agents for use in a combination therapy according to the present disclosure include cyclosporin, FK506, rapamycin, mycophenolic acid. Salicylates for use in said combination therapy include aspirin, sodium salicylate, choline salicylate and magnesium salicylate. Microbicidal agents include quinine and chloroquine.

Thus, in one embodiment the anti-inflammatory agent is a steroid, for example dexamethasone.

In one embodiment the therapy according to the present disclosure is employed in combination with paracetamol.

In one embodiment, the second therapy is a pan-Her inhibitor.

In one embodiment the DHODH inhibitor and a second therapy, such as a pan-HER inhibitor (such as particular Varlitinib) are administered sequentially in a treatment regimen, for example are administered on the same day.

In one embodiment the pan-HER inhibitor is a compound of formula (I):

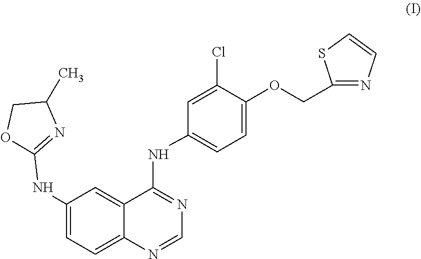 [SMILES: CC1COC(Nc2ccc3ncnc(Nc4ccc(OCc5nccs5)c(Cl)c4)c3c2)=N1]

an enantiomer thereof or a pharmaceutically acceptable salt of any one of the same.

In one embodiment the pan-HER inhibitor is Varlitinib:

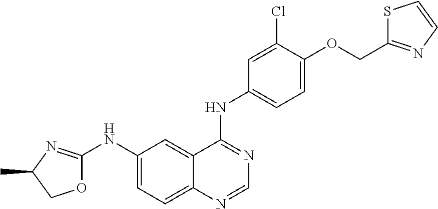 [SMILES: C[C@@H]1COC(Nc2ccc3ncnc(Nc4ccc(OCc5nccs5)c(Cl)c4)c3c2)=N1]

or a pharmaceutically acceptable salt thereof.

In one embodiment Varlitinib is employed as a free base.

Varlitinib at an appropriate dose is capable of inhibiting HER1, HER2 and HER4 directly and thought to be capable of inhibiting HER3 indirectly.

In one embodiment each dose of the compound of formula (I), (including Varlitinib) is in the range 100 to 900 mg, for example each dose is in the range of 300 to 500 mg, such as 400 mg, for example administered once or twice daily, such as twice daily.

In some instances, patients may benefit from having the initial dose reduced to 300 mg or 200 mg bi-daily.

Other patients may benefit from receiving the compound of formula (I), such as Varlitinib in a regime which is non-continuous, for example taking medication on alternate days instead of each day or taking medication for four sequential days followed by one, two or three days without medication.

In one embodiment the compound of formula (I), (including Varlitinib) is administered orally.

In one embodiment ASLAN003 is employed in combination with aspirin or warfarin.

### FORMULATIONS

In one embodiment the DHODH inhibitor is provided as a pharmaceutical formulation.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes.

In one embodiment the pharmaceutical formulation is for oral administration, for example formulated as tablets, pills, dragees, capsules, liquids, gels, syrups, slurries and suspensions, for ingestion by the patient.

Excipients may include lactose, dextrin, glucose, sucrose, sorbitol, starch, sugars, sugar alcohols and cellulose.

Other suitable forms for administration include parenteral administration, for example injection or infusion, such as bolus injection or continuous infusion.

Where the product is for injection or infusion, it may take the form of a suspension, solution or emulsion in an oily or aqueous vehicle and it may contain formulatory agents, such as suspending, preservative, stabilising and/or dispersing agents. Alternatively, the molecule may be in dry form, for reconstitution before use with an appropriate sterile liquid. Pharmaceutically acceptable carriers in therapeutic compositions may additionally contain liquids such as water, saline, glycerol and ethanol. Additionally, auxiliary substances, such as wetting agent, emulsifying agents, lubricant or pH buffering substances, may be present in such compositions.

A thorough discussion of pharmaceutically acceptable carriers is available in Remington's Pharmaceutical Sciences (Mack Publishing Company, N.J. 1991).

Comprising in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The background section of the present specification may be employed as basis for amendment to the claims because it contains important technical information.

The present application claims priority from SG 10202008397U, filed 31 Aug. 2021. The contents of the same is incorporated by reference. This disclosure may be employed as basis for making corrections to the present specification.

The invention will now be described with reference to the following examples, which are merely illustrative and should not be construed as limiting the scope of the present invention.

### EXAMPLES

### Example 1 Cell-Based Anti-SARS-CoV-2 Activity and Cell Toxicity for ASLAN003

### SARS-CoV-2 Assay

ASLAN003 was employed in a SARS-CoV-2 reporter assay using serial dilutions of the compound. 12k cells (A549-hACE2) were added per well in a 96-well plate in phenol-red free medium containing 2% FBS. On the next day, 2-fold serial dilutions of compounds were prepared in DMSO. The compounds were further diluted 100-fold in the phenol-red free culture medium containing 2% FBS. Cell culture fluids were removed and incubated with 50 μL of diluted compound solutions and 50 μL of SARS-CoV-2-Nluc viruses (MOI 0.025). At 48 h post-infection, 50 μL Nano luciferase substrates (Promega) were added to each well. Luciferase signals were measured using Synergy™ Neo2 microplate reader. The relative luciferase signals were calculated by normalizing the luciferase signals of the compound-treated groups to that of the DMSO-treated groups (set as 100%). The relative luciferase signal versus the log10 values of compound concentration was plotted in software Prism 8. The EC50 were calculated using a nonlinear regression model.

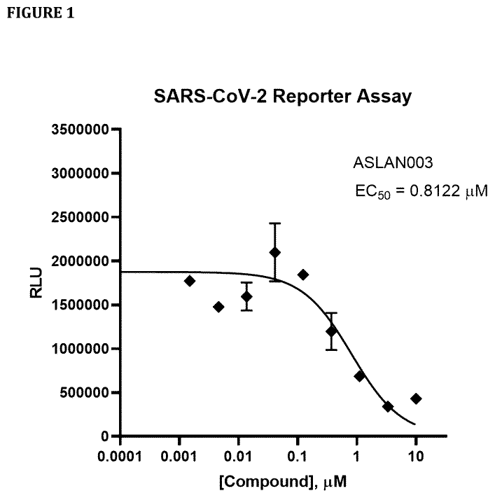

The results are shown in FIG. 1 and also summarized in Table 1.

### Cell Toxicity Study in A549 NSCLC Cell Line Using Cell TiterGlo Assay (Promega)

12k cells (A549) per well in a 96-well plate. The compounds are added on the next day. 48 hrs treatment. 3-fold serial dilution for ASLAN 003 starting from 10 μM.

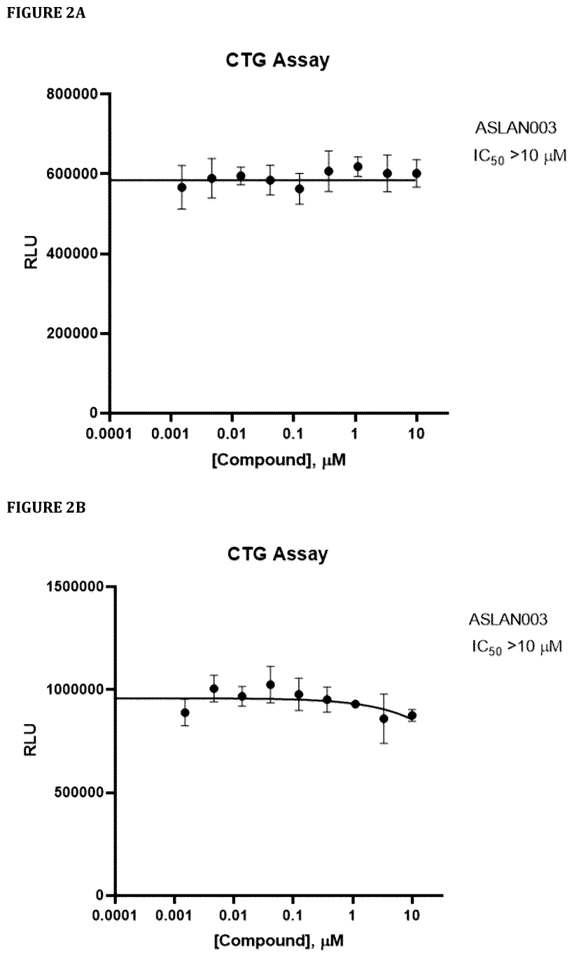

The results are shown in FIG. 2 and summarized in Table 1.

TABLE 1

| | SARS-COV-2 | Cell Cytotoxity | Cell Cytotoxity |
|---|---|---|---|
| | Reporter Assay | Assay 1 | Assay 2 |
| Compound | (IC₅₀, μM) | (IC₅₀, μM) | (IC₅₀, μM) |
| ASLAN003 | 0.8122 | >10 | >10 |

### Example 2—Dengue Virus and Zika Reporter Assays

ASLAN003 was employed in a Dengue virus reporter assay using serial dilutions of the compound. 2×10⁴ Huh7 cells were added per well in a 96-well plate and left overnight at 37° C., 5% CO₂. ASLAN003 was dissolved to 20 mM in DMSO and then diluted in 100% DMSO to 8 concentrations ranging from 4.8 μM to 625 μM in 1:2 step dilutions. ASLAN003 or DMSO was subsequently diluted 1:100 in DMEM culture medium with 3% FBS and added to cells lh before infection. The final concentration of DMSO in the assay was 1%. Cells were infected with Dengue virus (serotypes 1, 2, 3 or 4) or Zika virus (multiplicity of infection of 1) for 72 hours and harvested for viral titer determination.

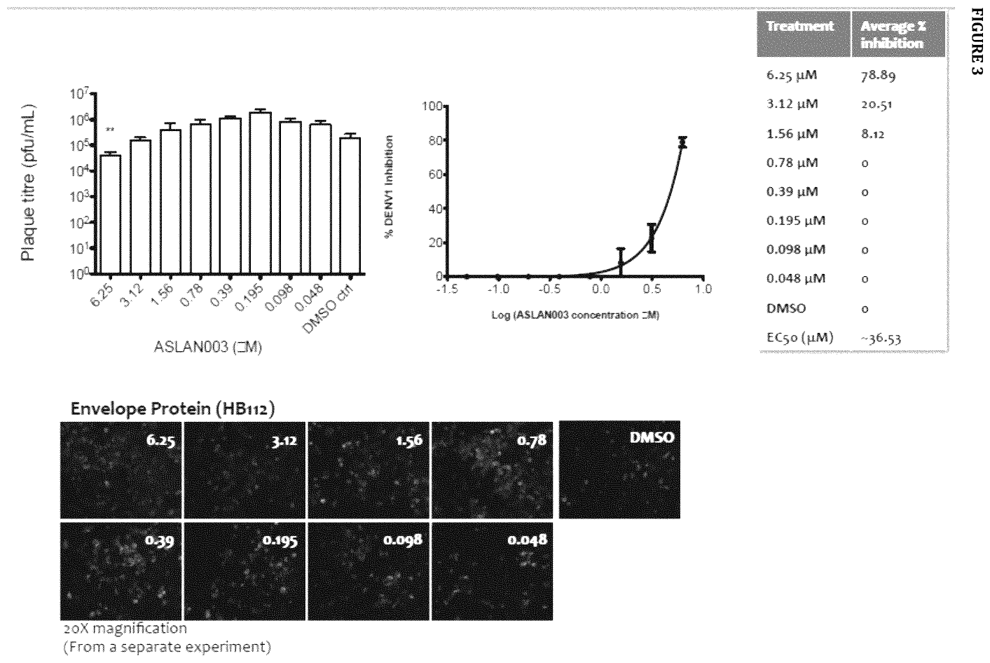

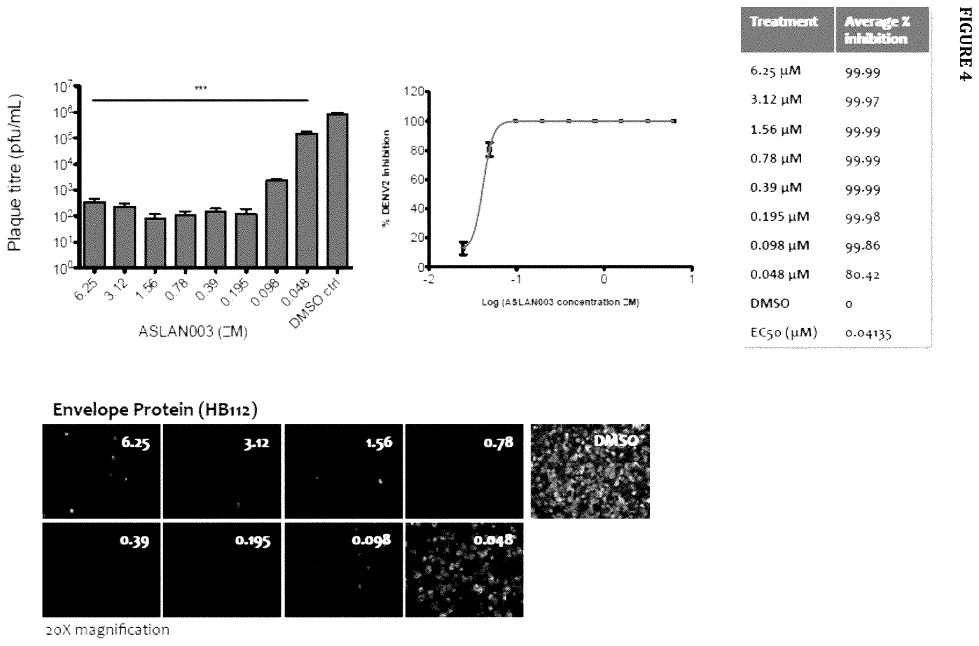

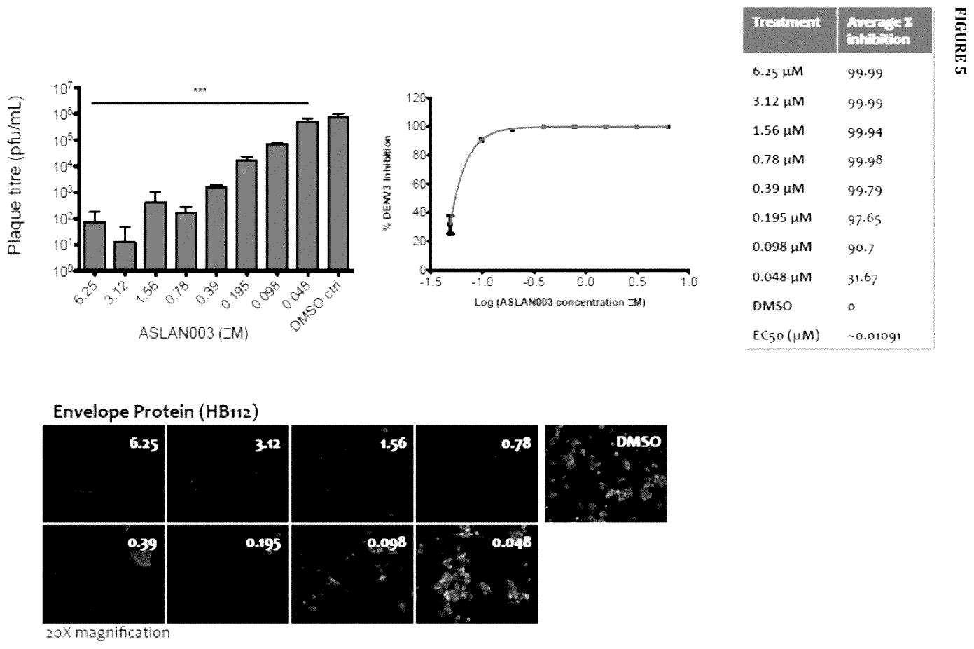

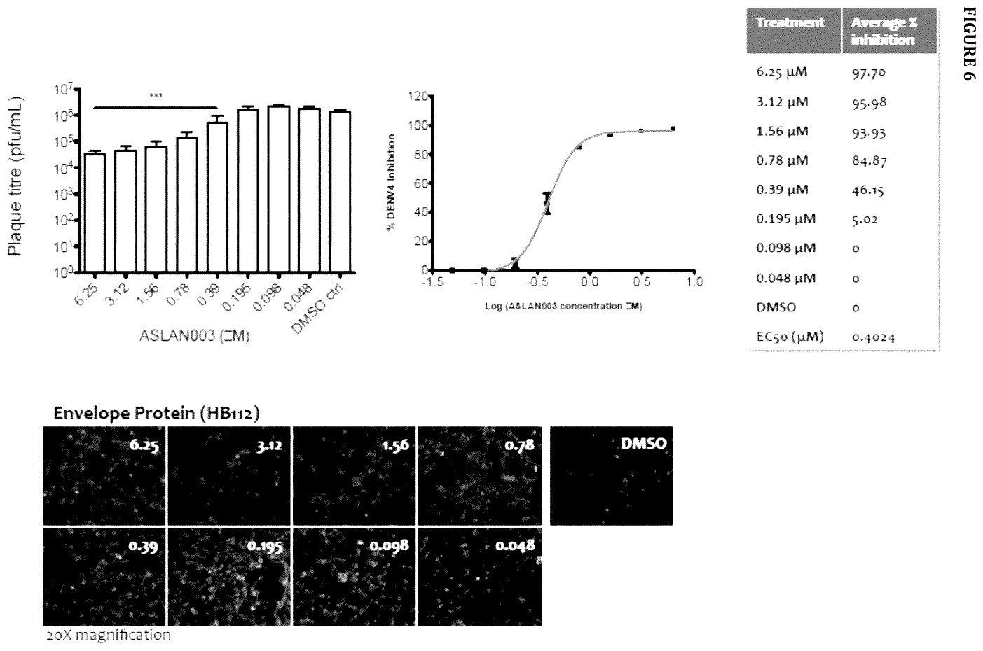

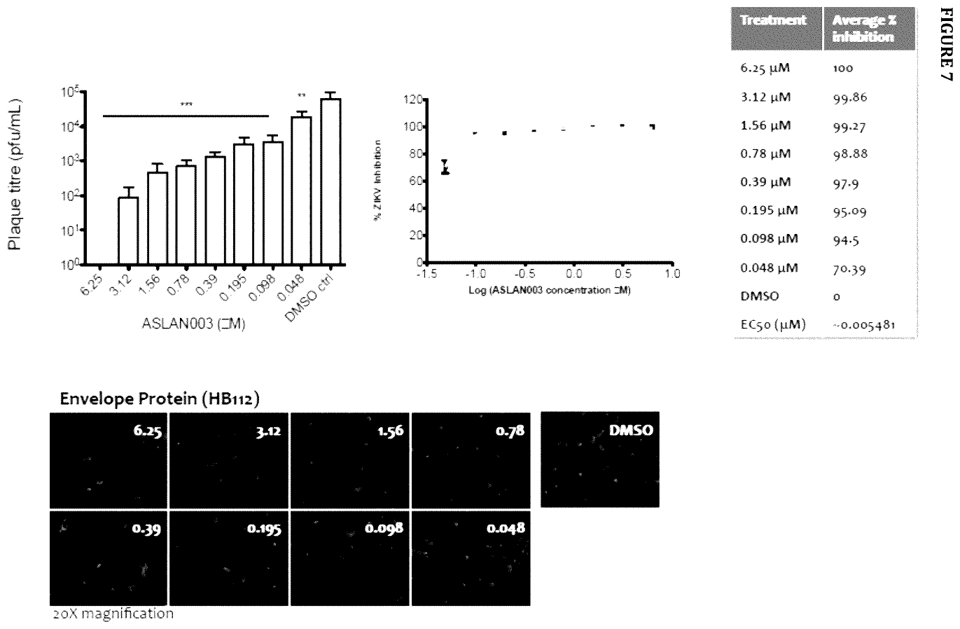

The results are shown in FIGS. 3 to 7.

### Example 3—Dengue Virus Time of Addition Assay

A time-of-addition assay for ASLAN003 was performed on Huh7 cells that were infected with Dengue virus serotype 2 (DENV2).

2×10⁴ Huh7 cells were added per well in a 96-well plate and left, overnight at 37° C., 5% CO₂. ASLAN003 was dissolved to 20 mM in DMSO and then diluted in 100% DMSO to 8 concentrations ranging from 4.8 μM to 625 μM in 1:2 step dilutions. ASLAN003 or DMSO was subsequently diluted 1:100 in DMEM culture medium with 3% FBS and added to cells 1 h before infection or 1/2/3/4 hours post infection. The final concentration of DMSO in the assay was 1%. Cells were infected with DENV2 (multiplicity of infection of 1) for 72 hours and harvested for viral titer determination.

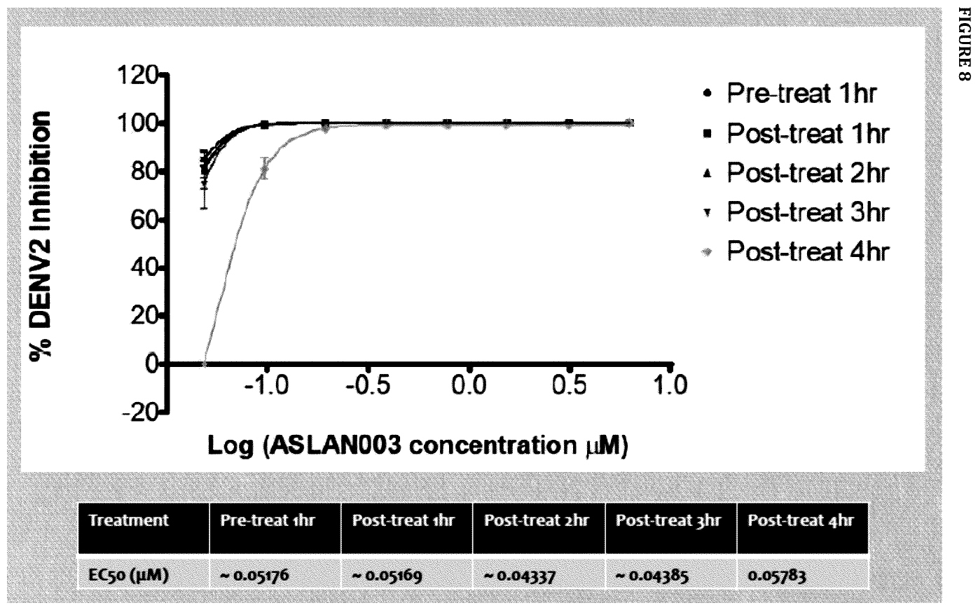

The results are shown in FIG. 8.

### Example 4—Chikungunya Virus Reporter Assays

ASLAN003 was employed in a Dengue virus reporter assay using serial dilutions of the compound. 2×10⁴ Huh7 cells were added per well in a 96-well plate and left overnight at 37° C., 5% CO₂. ASLAN003 was dissolved to 20 mM in DMSO and then diluted in 100% DMSO to 8 concentrations ranging from 4.8 μM to 625 μM in 1:2 step dilutions. ASLAN003 or DMSO was subsequently diluted 1:100 in DMEM culture medium with 3% FBS and added to cells 1 h before infection. The final concentration of DMSO in the assay was 1%. Cells were infected with CHIKV (multiplicity of infection of 1) for 72 hours and harvested for viral titer determination.

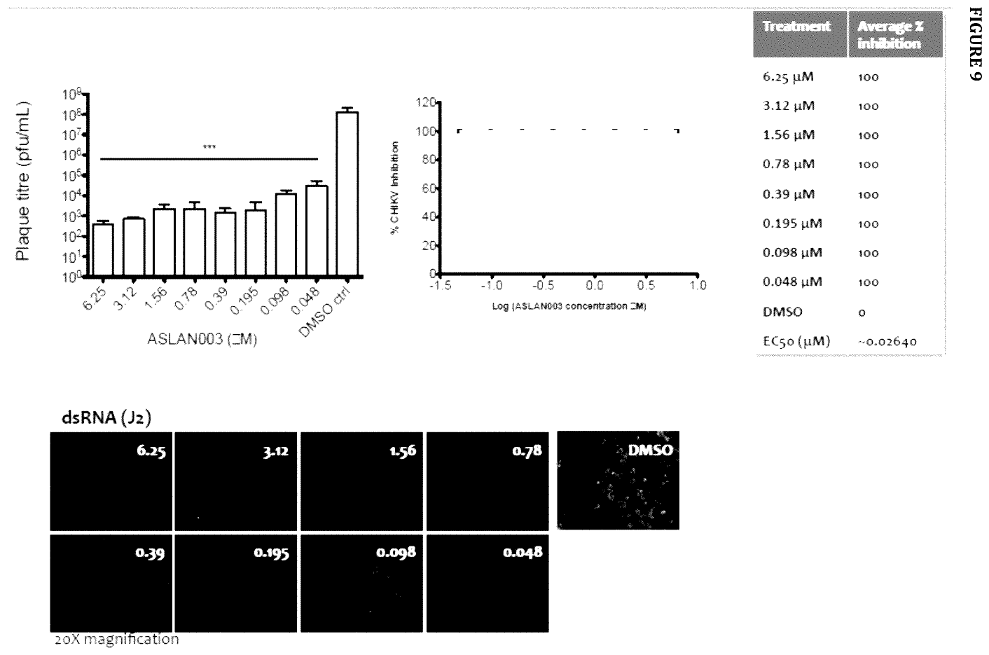

The results are shown in FIGS. 9 & 10.

### Example 5—Virucidal Effectiveness of ASLAN003 Against SARS-CoV-2

The objective of this study was to ascertain if ASLAN003 is effective in inhibiting SARS-CoV-2 in cell based assays using Vero E6 cells.

### Materials

Cell lines: Vero E6
Virus strain: SARS-CoV-2 (hCoV-19/Singapore/10/2020)
Drug/Compound: ASLAN003
Culture media: DMEM with 2% FCS

### Methods

Cell Viability Assay—To determine the cell viability profile of the ASLAN003, Vero E6 cells were seeded into 96-well plates at seeding densities of 1×10⁴ cells per well, respectively. Vero E6 cells were treated with the above compounds ata range of concentrations (0.0001, 0.001, 0.01, 1,10, 30, 50, and 100 μM) and incubated for 4 days. After incubation, the media was removed from the plates and washed once with PBS before addition of alamarBlue Cell Viability Reagent (Thermo Fisher Scientific) diluted 1:10 in media with 2% FCS. Fluorescence was detected after 2.5 h incubation at an excitation wavelength of 570 nm and an emission wavelength of 600 nm on an Infinite 200 Pro multiplate reader (Tecan). Data obtained from compound-treated cells and vehicle-treated cells were normalised against those obtained from untreated cells.

Dose-dependent Inhibition Assays—Vero E6 cells were seeded into 96-well plates and incubated overnight prior to drug inhibitory assays. Vero E6 cells were infected with SARS-CoV-2 at MOI of 1 for 1 hat 37° C. and then incubated with the compounds at working concentrations of 0.0001, 0.001, 0.01, 1, 10, 30, 50, and 100 μM. All dose-dependent inhibition assay plates were incubated for 4 days at 37° C., 5% CO₂, prior to harvesting of viral supernatants for virus titration.

Plaque Assay—To determine the virus titer, viral supernatants harvested were 10-fold serially diluted in DMEM. 200 μL of each serial diluted supernatant were applied to confluent Vero E6 cells. After 1 h of absorption, the inoculum was removed and 500 μl of 0.5% agarose overlay was added to each well and incubated for 3 days at 37° C., 5% CO2 to facilitate plaque formation. The cells were fixed with formalin overnight and the agarose was removed before staining with crystal violet for 5 minutes. The number of plaques were counted, and the virus titer of individual samples were expressed in the logarithm of plaque forming units (PFU) per mL.

StatisticalAnalyses—One-way analyses of variance (ANOVA) was used to evaluate the statistical significance of data obtained. Drug-treated samples expressing statistical difference when compared to control samples were subsequently subjected to a Dunnett's post-test, with * denoting that p<0.05 and *** denoting that p<0.001.

### Results

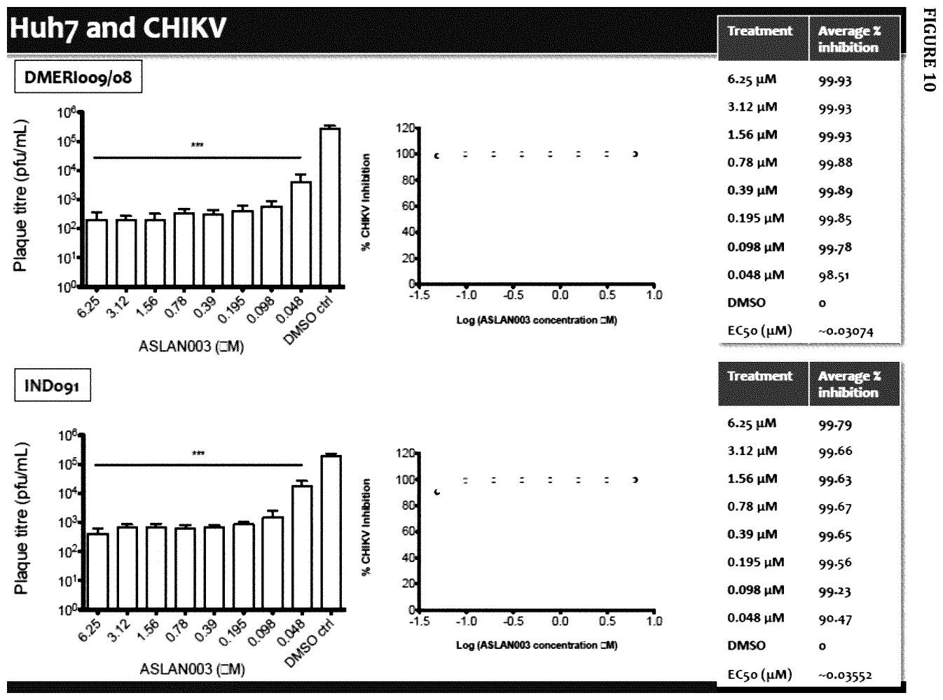

The potential antiviral activities of ASLAN003 on SARS-CoV-2 are shown in FIG. 10. Dosage dependent inhibition of SARS-CoV-2 are observed in Vero E6 cells treated with ASLAN003 and post virus infection. The EC and CC values ASLAN003 are calculated and displayed in Table 2.

TABLE 2
CC₅₀, EC₅₀, EC₉₀, and Selectivity Index (SI)
of ASLAN003 carried out on Vero E6 cells.

| | CC₅₀ | EC₅₀ | EC₉₀ | SI |
|---|---|---|---|---|
| ASLAN003 | 87.23 | 0.00142 | 0.00388 | 61,429.58 |

In conclusion, the experiments provide evidence that ASLAN003 has the potential to inhibit the replication of SARS-CoV-2, Dengue virus, Zika virus and Chikungunya virus. This demonstrates the potential for ASLAN003 to be employed as an anti-viral agent.

1. A method of treating or preventing a viral infection, comprising administering a therapeutically effective amount of a DHODH inhibitor 2-(3,5-difluoro-3′methoxybiphenyl-4-ylamino)nicotinic acid or a pharmaceutically acceptable salt thereof to a patient in need thereof.

2. A method according to claim 1, wherein the method is for treating a patient with or suspected of having a viral infection.

3. A method according to claim 1, wherein the virus is in the family Coronaviridae.

4. A method according to claim 3, wherein the virus is selected from Sarbecovirus and Merbecovirus.

5. A method according to claim 4, where the virus is a Sarbecovirus.

6. A method according to claim 5, wherein, the virus is selected from SARS-CoV and SARS-CoV-2, such as a SARS-CoV2.

7. A method according to claim 6, wherein the virus is MERS-CoV.

8. A method according to claim 1, wherein the virus is in the family Flaviviridae.

9. A method according to claim 8, wherein the virus is from the genus Flavivirus.

10. A method according to claim 8, wherein the virus is selected from Dengue virus and Zika virus.

11. A method according to claim 8, wherein the virus is a Dengue virus, for example Dengue virus serotype 1, 2, 3 or 4.

12. A method according to claim 8, wherein the virus is Zika virus.

13. A method according to claim 1, wherein the virus is in the genus alphavirus.

14. A method according to claim 13, wherein the virus is selected from Chikungunya virus, Bebaru virus, Getah virus, Mayaro virus, O'nyong'nyong virus, Ross River virus and Semliki Forest virus.

15. A method according to claim 13, wherein the virus is Chikungunya virus.

16. A method according to claim 1, wherein the virus is selected from the group comprising a Sarbecovirus and a mosquito borne virus.

17. A method according to claim 16, wherein the Sarbecovirus is SARS-CoV or SARS-CoV-2, in particular SARS-CoV-2.

18. A method according to claim 16, wherein the mosquito borne virus is selected from the group comprising Dengue (such as Dengue serotype 1, 2, 3 or 4), Zika and Chikungunya.

19. A method according to claim 1, wherein the virus is selected from the group comprising SARS-CoV-2, Dengue (such as Dengue serotype 1, 2, 3 or 4), Zika and Chikungunya.

20. A method according to claim 1, wherein the patient has co-morbidities.

21. A method according to claim 20, wherein the co-morbidity is selected from obesity, asthma, COPD, diabetes, kidney failure, heart disease (including heart failure), cancer, dementia, liver disease, and combinations thereof.

22. A method according to claim 1, wherein the DHODH inhibitor is employed in combination with a further therapy.

23. A method according to claim 22, wherein the further therapy is an anti-viral and/or an anti-inflammatory.

24. A method according to claim 23, wherein the anti-inflammatory is a steroid, such as dexamethasone.

25. A method according to claim 23, wherein the anti-viral is remdesivir.

26. A method according to claim 1, wherein the DHODH inhibitor is administered at a dose in the range 1 mg to 400 mg per day, such as 100 mg to 400 mg per day.

27. (canceled)

28. (canceled)
